(19) United States
(12) Patent Application Publication
(10) Pub. No.: US 2006/0258968 A1
(43) Pub. Date: Nov. 16, 2006

(54) Appartaus and method for applying cranial occipital pressure

(76) Inventor: John P. Slater II, Davis, OK (US)

Correspondence Address: DUNLAP, CODDING & ROGERS P.C., PO BOX 16370, OKLAHOMA CITY, OK, 73113, US

(21) Appl. No.: 11/402,013

(22) Filed: Apr. 11, 2006

Related U.S. Application Data
(60) Provisional application No. 60/669,997, filed on Apr. 11, 2005.

(51) Int. Cl. A61F 5/00

(52) U.S. Cl. 602/32

(57) ABSTRACT

An apparatus and method for applying occipital pressure and stretching the spinal column of an individual having a body and a pair of projections extending from the body such that the projections are positionable against the occiput of the individual. The apparatus further includes a head connector attached to the body and positionable about the head of the individual so as to secure the body to the individual's head with the projections positioned against the occiput of the individual. A support member is attached to the body and is connectable to a support structure to permit the body to be suspended from the support structure and support at least a portion of the weight of the individual to place the spinal column of the individual in tension.

### CROSS REFERENCE TO RELATED APPLICATIONS

This application claims benefit of U.S. Provisional Application No. 60/669,997, filed Apr. 11, 2005, which is incorporated herein by reference in its entirety.

### BACKGROUND OF THE INVENTION

A common cause of tension-type headaches is muscle contraction in the head, neck, or shoulders. Lengthy or extreme muscle tension in the head, neck, and shoulders may cause distortion in the alignment of cranial bones, leading to reduced flow of necessary cranial fluid to the area. Lack of cranial fluid tends to cause further discomfort in the head and neck. Relief from this type of discomfort may be obtained through stretching the neck muscles to reduce contractions, and through the application of pressure to the occipital region at the base of the skull. Application of pressure to the occiput send messages down the spinal column to relax all the muscles. Previous devices have been suggested for applying occipital pressure, and additional methods and devices have been suggested for applying tension to stretch the neck and back. However, it is desirable to apply tension to stretch the neck and back, while applying pressure to the occiput simultaneously. It is also desirable for this to be achievable outside of a hospital or clinic, and without requiring the assistance of a therapist or other individual.

### SUMMARY OF THE INVENTION

The present invention is directed to an apparatus and method for relieving discomfort in the head, neck, or back. This is done through application of cranial occipital pressure and by stretching the spinal column. Cranial occipital pressure may be applied by first positioning the apparatus so that adjustable projections in the apparatus engage the occiput, and then applying pressure to the occiput. Occipital pressure may be applied in conjunction with stretching the spinal column, or occipital pressure may be applied independently. For occipital pressure application, the apparatus may be used in any number of positions including a horizontal position, such as on the ground or on a bed, and a vertical position, such as on a door. In a horizontal position, the pressure results from the weight of a user's head on the apparatus. In a vertical position, the pressure results from the weight of a user's body when suspended from the apparatus.

For stretching the spinal column, the apparatus is typically used in the vertical position. The apparatus is secured to a point above a user and the user's head is positioned within the apparatus. The user then bends her knees. As the user lowers her lower body, her head remains at a fixed elevation, pressure is exerted on her occiput, and her spinal column is stretched and lengthened into a more comfortable position, stretching muscles and relieving spinal and neck compression.

### BRIEF DESCRIPTION OF THE DRAWINGS

FIG. 1 is a side elevational view of an apparatus constructed in accordance with the present invention shown attached to an individual's head.

FIG. 2 is a front elevational view of the apparatus of the present invention.

FIG. 3 is a perspective view of the apparatus of the present invention.

### DETAILED DESCRIPTION OF THE INVENTION

Referring now to the drawings, shown therein is an apparatus 10 for applying cranial occipital pressure and stretching the spinal column of an individual 11. The apparatus 10 has a body 12, projections 14 supported by the body 12, a support member 16 connected to the body 12 for suspending the body 12 from a support structure (not shown), and a head connector 18 for securing the body 12 and the projections 14 to the user's head. When secured to the user's head and supported from a support structure, the body 12 and the projections 14 cooperate to apply pressure to the base of the user's skull, while permitting the user to partially suspend herself from the apparatus 10 by bending her knees and thereby applying a tensile force to her spinal column.

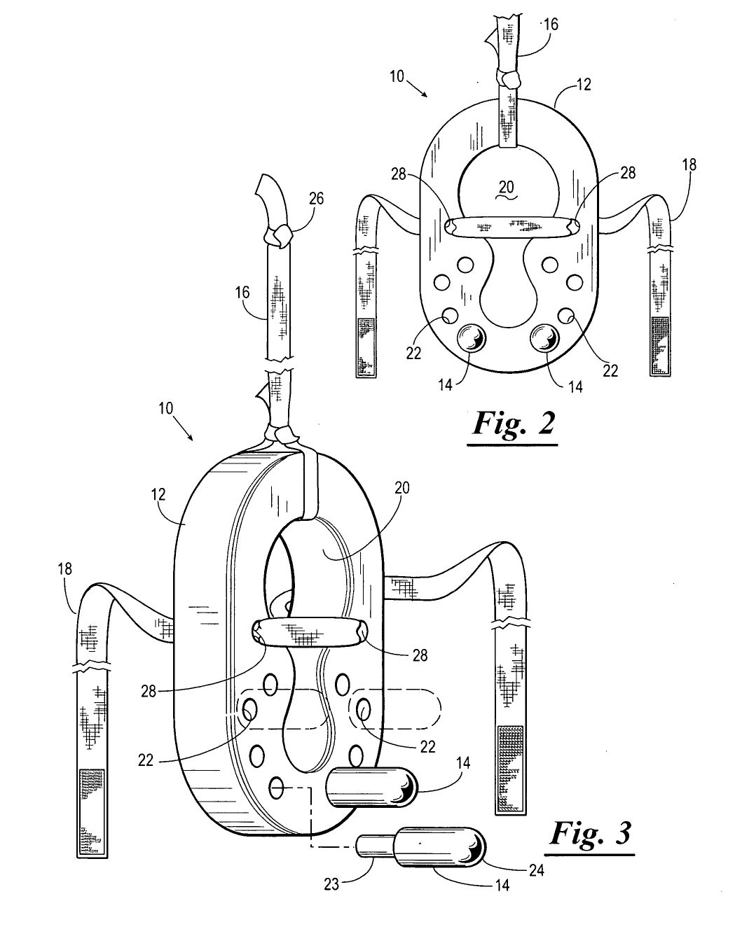

Referring now to FIGS. 2 and 3, the body 12 is shown to be a generally oval or elliptical shaped member having a head opening 20 and a plurality of projection receiving holes 22. The body 12 serves to connect the support member 16 to the head connector 18 and the projections 14. The body 12 may be made of any rigid material such as plastic, wood, or metal.

The head opening 20 is shaped such that the occiput of the individual rests on the projections 14 while the back of the user's head rests within the head opening 20. As shown in FIGS. 2 and 3, the head opening 20 is generally keyhole shaped. However, the head opening 20 may be formed in any shape suitable for stabilizing the body 12 on the user's head.

The projection receiving holes 22 are positioned near the bottom of the body 12 such that the projections 14 may engage the occiput of the user. The projection receiving holes 22 serve to hold the projections 14 in place while the apparatus 10 is in use. The projection receiving holes 22 are shown to be circular and sized to receive a portion of the projections 14. However, the projection receiving holes 22 may be formed in any shape so long as they are mateable with the projections 14. The projection receiving holes 22 allow the projections 14 to be positioned to accommodate various head and neck sizes, shapes, and positions.

The body 12 has four sets of projection receiving holes 22 (eight holes total) to accommodate various neck widths. While multiple projection receiving holes 22 are shown, only two projection receiving holes 22 are required. In addition to the projection receiving holes 22, customized placement of the projections 14 may be accomplished any number of ways, including but not limited to hook and loop fasteners, glue, hooks, or any other suitable connector. Additionally, the projections 14 may be fixedly attached to the body 12 or formed integrally with the body 12.

The projections 14 serve to apply pressure to the occiput or other desired pressure points on the user's head. A proximal end 23 of the projections 14 fits within one of the projection receiving holes 22, and a distal end 24 of each of the projections 14 extends outwardly from the body 12. The projections 14 are elongated, with the proximal 23 end shaped to be matingly received in the projection receiving holes 22, and the distal end 24 is preferably rounded to comfortably engage the occiput. However, it should be understood that the projections 14 may be formed in a variety of shapes, such as globular, pyramidal, or cuboidal. The projections 14 are shown to be made of a metal, such as stainless steel. However, other rigid materials, such as plastic or wood are also suitable.

The support member 16 serves to suspend the body 12 from a support structure (not shown). The support structure may be a door frame, a ceiling, a wall, or any other structure capable of supporting part of the user's weight. The support member 16 is a strap of material, such as nylon, wherein one end of the strap is secured to the body 12, and the other end of the strap is provided with a knot 26 to permit the strap to be placed between a door and a door frame (not shown). The knot 26 acts as an anchor to prevent the strap from being pulled through the space between the door and the door frame when weight is applied. The support member 16 may alternatively be provided with a hook (not shown) which engages the top of a door.

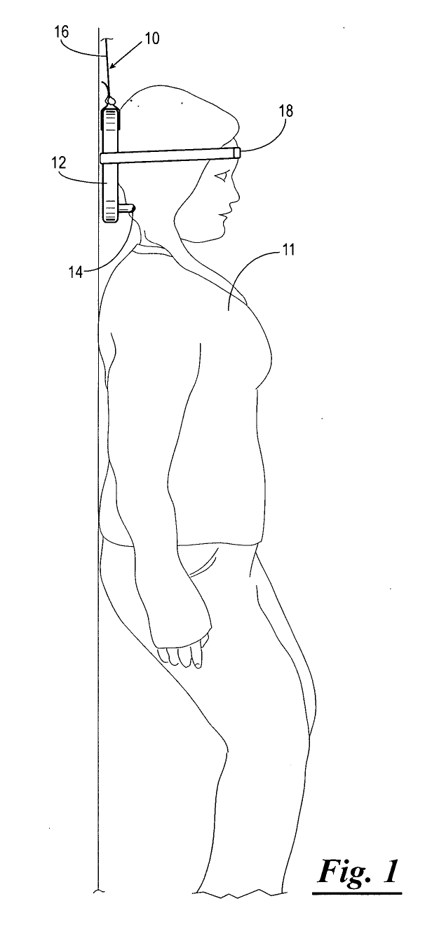

The head connector 18 serves to maintain the position of the user's head within the body 12, allowing the projections 14 to maintain pressure on the desired locations of the user's head. As shown in FIG. 1, the head connector 18 is attached to the body 12 near the top of the head opening 20 such that a user's head may be secured about the forehead. The head connector 18 may be a strap of material, such as nylon, with free ends that may be adjustably secured about the user's forehead. The head connector 18 is threaded through connector holes 28 in the body 12, wrapped back around the body 12 and secured around the user's head with a connector, such as hook and loop fasteners. While a single strap is shown, the head connector 18 may be any structure capable of securing the user's head to the body 12 of the apparatus 10, such as multiple straps, a cap or a cord.

Typically, the apparatus 10 is attached to a support structure before the user's head is secured to the apparatus 10. In the illustrated embodiment, this is done by hanging the knotted end of the support member 16 over a door and closing the door. This may generally be done by a single user, without assistance.

Once the apparatus 10 is attached to a support structure, the projections 14 are positioned in the projection receiving holes 22 that correspond closest to size of the width of the neck. The user's head is next secured to the apparatus 10, as shown in FIG. 1. The user's head is placed in the head opening 20 of the body 12 with the occiput resting on the projections 14. The head connector 18 is then secured around the head of the user. It will be appreciated that one might also secured the body 12 to the user's head with the head connector 18 prior to suspending the body 12 from a support structure.

FIG. 1 shows that when the user's head is secured to the apparatus 10, the head connector 18 works in conjunction with the projections 14 to substantially immobilize the head. The projections 14 rest against the occiput, preventing downward movement of the user's head, and allowing the neck muscles to stretch. The head connector 18 keeps the upper portion of the user's head in place, preventing the user's head from slipping out of the apparatus 10, and keeping the user's occiput against the projections 14. A portion of the back of the user's head rests on the body 12 of the apparatus 10, and another portion of the back of the user's head may rest within the head opening 20.

Once the apparatus 10 is supported by the support structure, the projections 14 are in place, and the user's head is secured to the body 12, the user slowly bends her knees. Because the user's head is at a fixed elevation, some of the support for the user's weight slowly shifts from her feet to her head. As this happens, the forces on the user's head, back, and neck switch from compressive to tensile. This allows the user's spinal column to be stretched, thereby relieving pressure caused by the compressive effect of gravity. Additionally, pressure is applied to the user's occiput, to cause an overall relaxation of the user's muscles. The user may bend her knees until an equalization point is found. The user may actively determine the most comfortable position by adjusting the bend in her knees.

From the above description, it is clear that the present invention is well adapted to carry out the objects and to attain the advantages mentioned herein, as well as those inherent in the invention. While a presently preferred embodiment of the invention has been described for purposes of this disclosure, it will be understood that numerous changes may be made which will readily suggest themselves to those skilled in the art and which are accomplished within the spirit of the invention disclosed and as defined in the appended claims.

What is claimed is:

1. An apparatus for applying occipital pressure and stretching the spinal column of an individual, comprising:
a body having a head receiving opening;
a pair of projections extending from the body such that the projections are positionable against the occiput of the individual when the individuals head is received in the head receiving opening of the body;
a head connector attached to the body and positionable about the head of the individual so as to secure the body to the individual's head with the projections positioned against the occiput of the individual; and
a support member having a one end connected to the body and another end connectable to a support structure to permit the body to be suspended from the support structure and support at least a portion of the weight of the individual to place the spinal column of the individual in tension.

2. The apparatus of claim 1 wherein the head receiving opening is substantially keyhole shaped.

3. The apparatus of claim 1 wherein the body has a plurality of pairs of projection receiving holes for removably receiving the projections, each pair of the projection receiving holes being spaced apart at different distances to permit the projections to be spaced apart at different distances.

4. The apparatus of claim 1 wherein each of the projections have a proximal end and a distal end, the distal end having a rounded shaped.

5. A method for applying occipital pressure and stretching the spinal column of an individual, comprising:
positioning a body having a pair of projections on the back of the individual's head so that the projections are positioned against the occiput of the individual;
suspending the body from a support structure;
attaching to the body to the individual's head with the projections positioned against the occiput of the individual; and
unloading weight from the individual's legs so as to transfer at least a portion of the individual's weight to the body and the projections and thereby place the spinal column of the individual in tension.

6. The method of claim 5 wherein the step of positioning the body on the back of the individual's head further comprises positioning a portion of the individual's head in a head receiving opening formed in the body.

7. The method of claim 5 further comprising adjusting the distance between the projections so that the projections substantially correspond to the width of the individual's neck.

8. The method of claim 5 wherein the step of unloading weight comprises bending the individual's knees.
